SEQUENCE LISTING (12 sequences)

SEQ ID NO: 1 - DNA, 743 bp, Homo Sapiens
  misc_feature: TSLP
  CDS (200)..(676)
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo Sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, Homo Sapiens
  MISC_FEATURE: LCDR1
GGNNLGSKSVH

SEQ ID NO: 4 - protein, 7 aa, Homo Sapiens
  MISC_FEATURE: LCDR2
DDSDRPS

SEQ ID NO: 5 - protein, 11 aa, Homo Sapiens
  MISC_FEATURE: LCDR3
QVWDSSSDHVV

SEQ ID NO: 6 - protein, 5 aa, Homo Sapiens
  MISC_FEATURE: HCDR1
TYGMH

SEQ ID NO: 7 - protein, 17 aa, Homo Sapiens
  MISC_FEATURE: HCDR2
VIWYDGSNKHYADSVKG

SEQ ID NO: 8 - protein, 13 aa, Homo Sapiens
  MISC_FEATURE: HCDR3
APQWELVHEAFDI

SEQ ID NO: 9 - DNA, 366 bp, Homo Sapiens
  misc_feature: Heavy Chain VH
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 122 aa, Homo Sapiens
  MISC_FEATURE: Heavy Chain VH
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, Homo Sapiens
  misc_feature: Light Chain VL
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 108 aa, Homo Sapiens
  MISC_FEATURE: Light Chain VL
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

(19) United States
(12) Patent Application Publication
(10) Pub. No.: US 2021/0148910 A1
(43) Pub. Date: May 20, 2021

(54) COMPOSITIONS AND METHODS FOR TREATMENT OF ATOPIC DERMATITIS AND TREATMENT SELECTION

(71) Applicant: MEDIMMUNE, LLC, GAITHERSBURG, MD (US)

(72) Inventor: MICHAEL D. HOWELL, GAITHERSBURG, MD (US)

(21) Appl. No.: 16/637,294

(22) PCT Filed: Aug. 15, 2018
(86) PCT No.: PCT/IB2018/056131

§ 371 Date: Feb. 7, 2020

Related U.S. Application Data
(60) Provisional application No. 62/546,210, filed on Aug. 16, 2017.

(51) Int. Cl. G01N 33/577; G01N 33/68

(52) U.S. Cl. CPC G01N 33/577; G01N 2800/202; G01N 33/6803

(57) ABSTRACT

The invention generally features compositions and methods for characterizing atopic dermatitis as responsive to anti-Thymic Stromal Lymphopoietin (TSLP) therapy by detecting alterations in the levels of polypeptide and polynucleotide markers present in patient samples, and related treatment methods.

### BACKGROUND OF THE INVENTION

Atopic dermatitis (also termed “AD”) is the most common chronic inflammatory skin disease affecting up to 25% children and 10% adults. Sufferers of atopic dermatitis have significantly impaired quality of life due to a vicious cycle of intense itching and scratching, insomnia, and/or depression and anxiety. Atopic dermatitis is believed to be caused by a complex interaction of genetic and environmental factors, which may explain why some treatments are effective in some atopic dermatitis patients but not others.

New methods of treatment and methods for predicting the responsiveness of atopic dermatitis patients to therapies are urgently required. Methods of characterizing atopic dermatitis have the potential to personalize treatment selection and to direct atopic dermatitis patients to effective therapies.

### SUMMARY OF THE INVENTION

As described below, the invention generally features compositions and methods for characterizing and treating atopic dermatitis, wherein the atopic dermatitis is found to be responsive to anti-Thymic Stromal Lymphopoietin (TSLP) therapy by detecting alterations in the levels of polypeptide and polynucleotide markers present in patient samples. Thymic stromal lymphopoietin is a protein belonging to the cytokine family. It is known to play an important role in the maturation of T cell populations through activation of antigen presenting cells. It may be encoded by the mRNA of SEQ ID NO: 1, whilst the full length amino acid sequence of TSLP is given in SEQ ID NO: 2.

In one aspect, the invention provides a method of treating a subject having atopic dermatitis, the method involving administering to the subject an agent that reduces the expression or activity of a Thymic Stromal Lymphopoietin (TSLP) polypeptide, where the subject is identified as having an increase in the level of Brain Derived Neurotrophin (BDNF) polypeptide in circulation or an increase in the level of Brain Derived Neurotrophin (BDNF) polynucleotide in a skin sample derived from the subject relative to a reference.

In another aspect, the invention provides a method of treating a subject having atopic dermatitis, the method involving administering to the subject an agent that reduces the expression or activity of a Thymic Stromal Lymphopoietin (TSLP) polypeptide, where the subject is identified as having an increase in the level of Brain Derived Neurotrophin (BDNF) polynucleotide and Amphiregulin polynucleotide in a skin sample derived from the subject relative to a reference.

In another aspect, the invention provides a method of treating a subject having atopic dermatitis, the method involving administering to the subject an agent that reduces the expression or activity of a Thymic stromal lymphopoietin (TSLP) polypeptide, where the subject is identified as having an increase in the level of Brain Derived Neurotrophin (BDNF) polynucleotide, Amphiregulin polynucleotide, and one or more of NTRK2, NTRK3, or NTF3 polynucleotides in a skin sample derived from the subject relative to a reference.

In another aspect, the invention provides a method of treating a subject having atopic dermatitis, the method involving administering to the subject an agent that reduces the expression or activity of a Thymic stromal lymphopoietin (TSLP) polypeptide, where the subject is identified as having an increase in the level of Brain Derived Neurotrophin (BDNF) polypeptide and an increase in CNTF and/or CNTFR in blood, plasma, or sera derived from the subject relative to a reference.

In another aspect, the invention provides a method of treating a subject having atopic dermatitis, the method involving administering to the subject an agent that reduces the expression or activity of a Thymic stromal lymphopoietin (TSLP) polypeptide, where the subject is identified as having an alteration in a biomarker polypeptide selected from the group consisting of Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3) in a blood, plasma, or sera sample of the subject relative to a reference, thereby treating the atopic dermatitis.

In another aspect, the invention provides a method of treating a subject having atopic dermatitis, the method involving administering to the subject an agent that reduces the expression or activity of a Thymic stromal lymphopoietin (TSLP) polypeptide, where the subject is identified as having an alteration in a biomarker polynucleotide selected from the group consisting of Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3) in a skin sample of the subject relative to a reference, thereby treating the atopic dermatitis.

In another aspect, the invention provides a method of identifying a subject as having atopic dermatitis (AD) responsive to an anti-TSLP therapy, the method involving detecting an increase in the level of Brain Derived Neurotrophic Factor (BDNF) polypeptide in circulation or an increase in the level of Brain Derived Neurotrophic Factor (BDNF) polynucleotide in a skin sample derived from the subject relative to a reference, thereby identifying the subject as having atopic dermatitis that is responsive to anti-TSLP therapy.

In another aspect, the invention provides a method of identifying a subject as having atopic dermatitis (AD) responsive to an anti-TSLP therapy, the method involving detecting an increase in the level of Brain Derived Neurotrophic Factor (BDNF) polynucleotide and Amphiregulin polynucleotide in a skin sample derived from the subject relative to a reference, thereby identifying the subject as having atopic dermatitis that is responsive to anti-TSLP therapy.

In another aspect, the invention provides a method of identifying a subject as having atopic dermatitis (AD) responsive to an anti-TSLP therapy, the method involving detecting an increase in the level of Brain Derived Neurotrophic Factor (BDNF) polynucleotide, Amphiregulin polynucleotide, and one or more of NTRK2, NTRK3, or NTF3 polynucleotides in a skin sample derived from the subject, thereby identifying the subject as having atopic dermatitis that is responsive to anti-TSLP therapy.

In another aspect, the invention provides a method of identifying a subject as having atopic dermatitis (AD) responsive to an anti-TSLP therapy, the method involving detecting an increase in the level of Brain Derived Neurotrophic Factor (BDNF) polypeptide and an increase in CNTF and/or CNTFR in blood, plasma, or sera derived from the subject, thereby identifying the subject as having atopic dermatitis that is responsive to anti-TSLP therapy.

In another aspect, the invention provides a method of identifying a subject as having atopic dermatitis (AD) responsive to an anti-TSLP therapy, the method involving detecting antibody binding to a circulating polypeptide marker selected from the group consisting of Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3) in a blood, plasma, or sera sample of the subject; and detecting an alteration in the level of said marker in the sample relative to a reference, thereby identifying the subject as having atopic dermatitis (AD) responsive to an anti-TSLP therapy.

In another aspect, the invention provides a method of identifying a subject as having atopic dermatitis (AD) responsive to an anti-TSLP therapy, the method involving detecting probe binding to a polynucleotide marker selected from the group consisting of Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3) in a skin sample of the subject; and detecting an alteration in the level of said marker in the sample relative to a reference, thereby identifying the subject as having atopic dermatitis (AD) responsive to an anti-TSLP therapy.

In another aspect, the invention provides a method of monitoring the efficacy of therapy in a subject, the method involving administering an anti-TSLP therapy to the subject; and detecting the level of Brain Derived Neurotrophic Factor polynucleotide in a skin sample derived from the subject relative to the level of Brain Derived Neurotrophic Factor polynucleotide in a skin sample obtained from the subject at an earlier point in time, wherein a decrease in the level of BDNF over time indicates that the anti-TSLP therapy is effective.

In another aspect, the invention provides a kit for the treatment of atopic dermatitis (AD), containing an agent that reduces the expression or activity of a Thymic stromal lymphopoietin (TSLP) polypeptide, and one or more of a capture molecule or probe that specifically binds a polypeptide or polynucleotide biomarker that is one or more of Amphiregulin (AREG), Ciliary Neurotrophic Factor (CNTF), Ciliary Neurotrophic Factor Receptor (CNTFR), Brain Derived Neurotrophin (BDNF), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), or Neurotrophic Tyrosine Kinase Receptor Type 3 (NTRK3).

In another aspect, the invention provides a kit for the treatment of atopic dermatitis (AD), containing an agent that reduces the expression or activity of a Thymic stromal lymphopoietin (TSLP) polypeptide, and one or more of a capture molecule or probe that specifically binds a polypeptide or polynucleotide biomarker that is one or more of Amphiregulin (AREG), Ciliary Neurotrophic Factor (CNTF), Ciliary Neurotrophic Factor Receptor (CNTFR), Brain Derived Neurotrophin (BDNF), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), or Neurotrophic Tyrosine Kinase Receptor Type 3 (NTRK3).

In various embodiments of any aspect delineated herein, BDNF polypeptide in circulation is measured in blood, plasma, or serum sample derived from the subject. In various embodiments of any aspect delineated herein, BDNF polynucleotide in skin is increased in a skin biopsy of lesional or non-lesional skin compared to a control sample. In various embodiments of any aspect delineated herein, the control sample is derived from a subject having atopic dermatitis that is not responsive to anti-TSLP therapy. In various embodiments of any aspect delineated herein, the control sample is derived from a healthy subject. In various embodiments of any aspect delineated herein, the method further involves detecting the level of amphiregulin polypeptide in the sera of the subject relative to the level present in sera of the subject at an earlier point in time, wherein an increase in said level over time indicates that the anti-TSLP therapy is effective.

In various embodiments of any aspect delineated herein, the method further involves detecting an increase in Ciliary Neurotrophic Factor (CNTF) polynucleotide or Ciliary Neurotrophic Factor Receptor (CNTFR) polynucleotide in circulation compared to a control sample. In various embodiments of any aspect delineated herein, the method further involves detecting an increase in amphiregulin polynucleotide in lesional and non-lesional skin biopsies. In various embodiments of any aspect delineated herein, the method further involves detecting an increase in a polynucleotide biomarker selected from the group consisting of NTRK2, NTRK3, and Neurotrophin Factor 3 (NTF3).

In various embodiments of any aspect delineated herein, the atopic dermatitis is responsive to treatment with the agent that reduces the expression or activity of a Thymic stromal lymphopoietin (TSLP) polypeptide. In various embodiments of any aspect delineated herein, the agent that reduces the expression or activity of the TSLP polypeptide is an anti-TSLP antibody, or antigen binding portion thereof.

In various embodiments, the anti-TSLP antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4 iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6 ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

In various embodiments, the anti-TSLP antibody comprises
a. a light chain variable domain selected from the group consisting of:
   i. a sequence of amino acids at least 80% identical to SEQ ID NO:12;
   ii. a sequence of amino acids encoded by a polynucleotide sequence that is at least 80% identical to SEQ ID NO:11;
   iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
   i. a sequence of amino acids that is at least 80% identical to SEQ ID NO:10;
   ii. a sequence of amino acids encoded by a polynucleotide sequence that is at least 80% identical to SEQ ID NO:9;
   iii. a sequence of amino acids encoded by a polynucleotide that hybridizes under moderately stringent conditions to the complement of a polynucleotide consisting of SEQ ID NO:9; or
c. a light chain variable domain of (a) and a heavy chain variable domain of (b), wherein the antibody specifically binds to a TSLP polypeptide as set forth in amino acids 29-159 of SEQ ID NO:2.

In various embodiments of any aspect delineated herein, the antibody is Tezepelumab (WHO Drug Information Vol. 30, No. 1, 2016 Recommended INN: List 75 pages 56-57).

In various embodiments of any aspect delineated herein, the subject is human. In various embodiments of any aspect delineated herein, the polypeptide is detected in an immunological assay. In various embodiments of any aspect delineated herein, the polynucleotide is detected by hybridization to a microarray or by gene expression analysis. In various embodiments of any aspect delineated herein, the reference is the level, expression, or activity of the corresponding polypeptide or nucleic acid molecule biomarker present in a control sample.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Thymic stromal lymphopoietin (TSLP) polypeptide” is meant a polypeptide or fragment thereof having at least about 85% or greater amino acid identity to the amino acid sequence provided at NCBI Accession No. NP_149024.1 (see SEQ ID NO: 2), and having TSLP biological activity. Exemplary TSLP biological activities include binding to TSLP receptor comprising CRLF2 and the IL-7R alpha chain.

By “Thymic stromal lymphopoietin (TSLP) nucleic acid molecule” is meant a polynucleotide encoding a TSLP polypeptide. An exemplary TSLP nucleic acid molecule is provided at NCBI Accession No.: AY037115.1 (SEQ ID NO: 1).

By “Ciliary neurotrophic factor (CNTF) polypeptide” is meant a polypeptide or fragment thereof having at least about 85% or greater amino acid identity to the amino acid sequence provided at NCBI Accession No. NP_000605, and having CNTF biological activity. Exemplary CNTF biological activities include binding to CNTF receptor and neurotrophic activity.

By “Ciliary neurotrophic factor (CNTF) nucleic acid molecule” is meant a polynucleotide encoding a CNTF polypeptide. An exemplary CNTF nucleic acid molecule is provided at NCBI Accession No. NM_000614.

By “Ciliary neurotrophic factor receptor (CNTFR) polypeptide” is meant a polypeptide or fragment thereof having at least about 85% or greater amino acid identity to the amino acid sequence provided at NCBI Accession No. NP_001193940, and having CNTFR biological activity. Exemplary CNTFR biological activities include binding to CNTF and neurotrophic activity.

By “Ciliary neurotrophic factor receptor (CNTFR) nucleic acid molecule” is meant a polynucleotide encoding a CNTFR polypeptide. An exemplary CNTFR nucleic acid molecule is provided at NCBI Accession No. NM_001842.

By “Brain-derived neurotrophic factor (BDNF) polypeptide” is meant a polypeptide or fragment thereof having at least about 85% or greater amino acid identity to the amino acid sequence provided at NCBI Accession No. NP_001137277, and having BDNF biological activity. Exemplary BDNF biological activities include binding to NTRK2 and neurotrophic activity.

By “Brain-derived neurotrophic factor (BDNF) nucleic acid molecule” is meant a polynucleotide encoding a BDNF polypeptide. An exemplary BDNF nucleic acid molecule is provided at NCBI Accession No. NM_001143805.

By “Nerve growth factor (NGF) polypeptide” is meant a polypeptide or fragment thereof having at least about 85% or greater amino acid identity to the amino acid sequence provided at NCBI Accession No. NP_002497, and having NGF biological activity. Exemplary NGF biological activities include binding to NTRK1 and neurotrophic activity.

By “Nerve growth factor (NGF) nucleic acid molecule” is meant a polynucleotide encoding a NGF polypeptide. An exemplary NGF nucleic acid molecule is provided at NCBI Accession No. NM_002506.

By “Neurotrophin 3 (NTF3) polypeptide” is meant a polypeptide or fragment thereof having at least about 85% or greater amino acid identity to the amino acid sequence provided at NCBI Accession No. NP_002518, and having NTF3 biological activity. Exemplary NTF3 biological activities include binding to NTRK3 and neurotrophic activity.

By “Neurotrophin 3 (NTF3) nucleic acid molecule” is meant a polynucleotide encoding a NTF3 polypeptide. An exemplary NTF3 nucleic acid molecule is provided at NCBI Accession No. NM_002527.

By “Neurotrophin 4 (NTF4) polypeptide” is meant a polypeptide or fragment thereof having at least about 85% or greater amino acid identity to the amino acid sequence provided at NCBI Accession No. NP_006170, and having NTF4 biological activity. Exemplary NTF4 biological activities include binding to NTRK2 and neurotrophic activity.

By “Neurotrophin 4 (NTF4) nucleic acid molecule” is meant a polynucleotide encoding a NTF4 polypeptide. An exemplary NTF4 nucleic acid molecule is provided at NCBI Accession No. NM_006179.

By “Neurotrophic Tyrosine Kinase Receptor Type 1 (NTRK1) polypeptide” is meant a polypeptide or fragment thereof having at least about 85% or greater amino acid identity to the amino acid sequence provided at NCBI Accession No. NP_002520, and having NTRK1 biological activity. Exemplary NTRK1 biological activities include binding to NGF and neurotrophic activity.

By “Neurotrophic Tyrosine Kinase Receptor Type 1 (NTRK1) nucleic acid molecule” is meant a polynucleotide encoding a NTRK1 polypeptide. An exemplary NTRK1 nucleic acid molecule is provided at NCBI Accession No. NM_002529.

By “Neurotrophic Tyrosine Kinase Receptor Type 2 (NTRK2) polypeptide” is meant a polypeptide or fragment thereof having at least about 85% or greater amino acid identity to the amino acid sequence provided at NCBI Accession No. NP_001007098, and having NTRK2 biological activity. Exemplary NTRK2 biological activities include binding to BDNF and/or NTF4 and neurotrophic activity.

By “Neurotrophic Tyrosine Kinase Receptor Type 2 (NTRK2) nucleic acid molecule” is meant a polynucleotide encoding a NTRK2 polypeptide. An exemplary NTRK2 nucleic acid molecule is provided at NCBI Accession No. NM_001007097.

By “Neurotrophic Tyrosine Kinase Receptor Type 3 (NTRK3) polypeptide” is meant a polypeptide or fragment thereof having at least about 85% or greater amino acid identity to the amino acid sequence provided at NCBI Accession No. NP_002521, and having NTRK3 biological activity. Exemplary NTRK3 biological activities include binding to NTF3 and neurotrophic activity.

By “Neurotrophic Tyrosine Kinase Receptor Type 3 (NTRK3) nucleic acid molecule” is meant a polynucleotide encoding a NTRK3 polypeptide. An exemplary NTRK3 nucleic acid molecule is provided at NCBI Accession No. NM_002530.

By “amphiregulin (AREG) polypeptide” is meant a protein having at least about 85% amino acid identity to NCBI Accession No. NP_001648 or a fragment thereof having T cell regulatory activity. The sequence of an exemplary amphiregulin polypeptide is provided at NCBI Accession No. NP_001648 By “amphiregulin (AREG) polynucleotide” is meant a polynucleotide that encodes an amphiregulin polypeptide.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)2, Fv, scFv, Fd, dAb, and other antibody fragments that retain antigen-binding function, i.e., the ability to bind a polypeptide specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” In particular embodiments, an antigen-binding domain comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies: Fab, Fab′, single chain V region fragments (scFv), fusion polypeptides, and unconventional antibodies.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have a specificity, an affinity, and/or a capability of interest (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has a specificity, an affinity, and/or a capability of interest.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FW regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

“Detect” refers to identifying the presence, absence or amount of the analyte to be detected. In various embodiments, the analyte is a polypeptide or nucleic acid biomarker.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. In a particular embodiment, a fragment of a polypeptide may contain 5, 10, 20, 30, 40, 50, 60, 70, 80, 90, 100, 200, or 300 amino acids.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences (see e.g., Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR).

By “increases” is meant a positive alteration. For example, an increase by at least 10%, 25%, 50%, 75%, 100%, 200%, 300%, 400%, 500%, 1000%, or more.

The term “isolated” refers to a molecule that is substantially free of other elements present in its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure.

By “reduces” is meant a negative alteration. For example, a reduction of 10%, 25%, 50%, 75%, or 100%.

By “reference” is meant a standard of comparison. In one embodiment, a reference level is the level, expression, or activity of a biomarker in a biological sample obtained from an unaffected tissue.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acid molecules, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant an agent (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity.

By “subject” is meant a mammal, including, but not limited to, a human or non-human mammal, such as a bovine, equine, canine, ovine, feline, or murine.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for an inflammatory or autoimmune disease or disorder according to the methods provided herein if the patient shows, e.g., total, partial, or transient alleviation or elimination of symptoms associated with the disease or disorder.

As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 includes two graphs depicting proteomic expression of CNTF and CNTFR at baseline, day 29 (D29) and day 85 (D85) in sera of atopic dermatitis patients administered Tezepelumab. Tezepelumab, which has a 30-day half-life, was administered at Day 1. At baseline, increased levels of CNTF and CNTFR were noted in sera of responding patients relative to non-responding patients. Patients responding to treatment with Tezepelumab are depicted with circles. Non-responders are depicted with squares. Graphs depict the quantification of fluorescence data obtained by microarray analysis.

FIG. 2 are graphs depicting proteomic expression of BDNF, NGF, NTF3, and NTF4 at baseline, day 29 (D29) and day 85 (D85) in sera of atopic dermatitis patients administered Tezepelumab. Significantly, a 50% reduction was observed in BDNF levels at day 29 in the sera of patients that responded to Tezepelumab therapy. No alterations were noted in levels of NTF3 and NTF4 in sera.

FIG. 3 are graphs depicting proteomic expression of NTRK1, NTRK2, and NTRK3 at baseline, day 29 (D29) and day 85 (D85) in sera of atopic dermatitis patients administered Tezepelumab. No significant changes in NTRK1, NTRK2, and NTRK3 levels were observed in sera.

FIG. 4 are graphs depicting genomic expression of BDNF. NTF3, and NTF4 in lesional (LS) and non-lesional (NL) skin biopsies of atopic dermatitis patients administered Tezepelumab or placebo at Day 1. Biopsies were obtained at Day 1 and Day 29. At baseline, levels of BDNF and NTF3 were increased in lesional skin biopsies of patients that were subsequently found to respond to Tezepelumab therapy relative to levels present at baseline in non-responding patients. This finding indicates that increased levels of BDNF and NTF in skin biopsies may be used as markers to identify patients likely to respond to Tezepelumab therapy. BDNF is linked to eosinophil survival. On Day 29, levels of BDNF were reduced in skin lesions of responding patients.

FIG. 5 are graphs depicting genomic expression of NTRK2 in lesional and non-lesional skin biopsies of atopic dermatitis patients administered Tezepelumab or placebo at Day 1. Baseline levels of NTRK2 genomic expression were increased in patients that were responsive to Tezepelumab relative to levels present at baseline in non-responding patients. These findings indicate that increased genomic expression of NTRK2 in lesional skin biopsies may be used as a marker to identify patients likely to respond to Tezepelumab therapy.

FIG. 6 are graphs depicting genomic expression of NTRK3 in lesional and non-lesional skin biopsies of atopic dermatitis patients administered Tezepelumab or placebo at Day 1. Baseline levels of NTRK3 were higher in subjects found to respond to Tezepelumab relative to levels present at baseline in non-responding patients. This indicates that increased levels of NTRK3 relative to a levels in lesional skin biopsies from patients treated with placebo may be used as a marker to identify patients likely to respond to Tezepelumab therapy.

FIG. 7 are graphs depicting correlations between protein levels of TSLP and BDNF, TSLP and NTF3, TSLP and NTF4/5, TSLP and AREG in the sera of atopic dermatitis patients

FIG. 8 are graphs depicting correlation between protein levels of TSLP and TrkA, TSLP and TrkB, TSLP and TrkC, and TSLP and TSLPR/CRLF2 in sera of atopic dermatitis patients.

FIG. 9 are graphs showing genomic expression of amphiregulin in lesional and non-lesional skin biopsies. At baseline, genomic expression of amphiregulin (probe 215564) is increased in lesional skin biopsies obtained from patients that ultimately responded to Tezepelumab therapy. Levels of Amphiregulin are reduced in skin lesions following treatment with Tezepelumab.

FIG. 10 is a graph showing that levels of amphiregulin at baseline are capable of separating those patients that will respond to Tezepelumab treatment.

FIG. 11 is a schematic diagram indicating that genomic expression of TSLP and other inflammatory mediators are increased in skin lesions and can be used to identify patients that are responsive to treatment with Tezepelumab; the diagram also shows that increased levels of TSLP and other inflammatory mediators are observed in the sera of patients likely to respond to Tezepelumab.

FIG. 12 is a table demonstrating the direct induction of AREG, BDNF, NGF, NTRK1/TrkA, and TSLPR/CRLF2 gene expression in eosinophils and basophils following stimulation with TSLP for 24 hours.

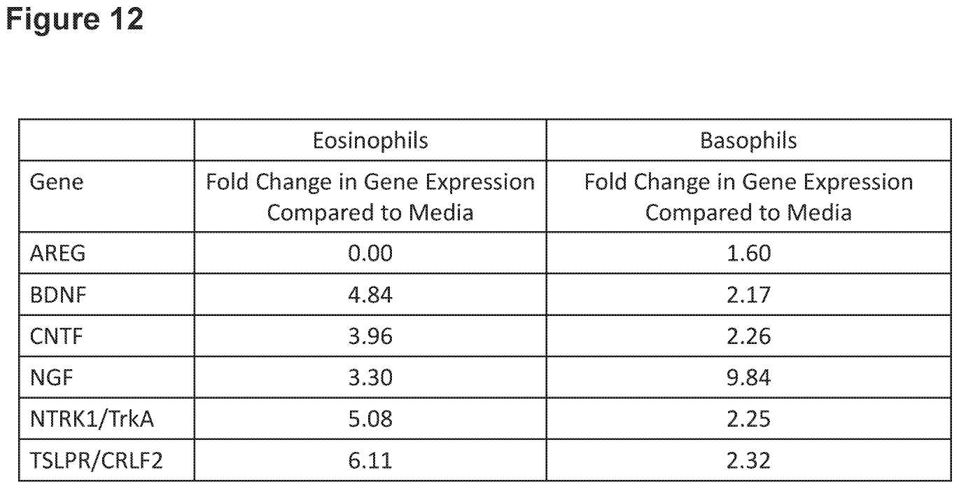

### DETAILED DESCRIPTION OF THE INVENTION

The invention generally features compositions and methods for characterizing atopic dermatitis as responsive to anti-Thymic Stromal Lymphopoietin (TSLP) therapy by detecting alterations in the levels of polypeptide and polynucleotide markers present in patient samples, and related treatment methods.

The invention is based, at least in part, on the discovery that patients responsive to Tezepelumab can be identified by characterizing levels of polypeptide and polynucleotide biomarkers (e.g., Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3)) in skin and serum samples obtained from the patients. BDNF and Amphiregulin generally correlate with TLSP levels, and may be measured in place of measuring TLSP.

In one aspect, the biomarkers in the present invention are for diagnostic use to aid in the identification of those individuals that would benefit from antagonism of TSLP (e.g., an anti-TSLP antibody). Cytokines that regulate the TH2 response include, for example, IL-33, IL-25, and/or TSLP, which drive IL-13 and IL-4 mediated immune response However, the cytokines are present in small quantities and hard to detect and measuring cytokines is expensive and impractical with current methods. As described herein, it has been discovered that expression levels of neurotrophic factor polypeptides and polynucleotides (e.g., BDNF, Amphiregulin, NTRK3) correlated with cytokine levels. Thus, soluble neurotrophic factors have the potential to serve as proxies for detecting the levels of one or more cytokines (TSLP, IL-33, IL-25, etc.). This allows for a personalized approach to atopic dermatitis therapy based on the results of a diagnostic assay, for example a point-of-care immunoassay or genomic expression assay, prior to commencing appropriate therapy.

Accordingly, the present invention provides methods for characterizing atopic dermatitis in a patient suffering from the disease, including the responsiveness of the patient's atopic dermatitis to available treatment for the disease, and methods for selecting an appropriate treatment for atopic dermatitis.

### Atopic Dermatitis

Atopic dermatitis is the most common chronic inflammatory skin disease affecting up to 25% of children and 10% of adults. Sufferers of atopic dermatitis have significantly impaired quality of life due to a vicious cycle of intense itching and scratching, insomnia, and/or depression and anxiety. Atopic dermatitis is believed to be caused by a complex interaction of genetic and environmental factors. Atopic dermatitis lesional skin is characterized by impaired protective barrier, deficient innate immune response, and predominantly Th2 mediated inflammation. Increased Th2 Axis is observed in atopic dermatitis skin and circulation. IL-4 and IL-13 expression is detected in non-lesional and lesional atopic dermatitis skin and increased IL-4 and IL-13 T-cells in atopic dermatitis. Additionally, atopic dermatitis sufferers have increased susceptibility to bacterial, viral, and fungal infections, for example, >90% of atopic dermatitis patients colonized with Staphylococcus aureus. Approximately 80% of atopic dermatitis patients have elevated serum IgE levels (>200 kU/L) and increased allergen specific responses.

Varied effectiveness of biologics targeting different inflammatory pathways highlights heterogeneity and complexity of atopic dermatitis. Without being bound by theory, responses in different atopic dermatitis patients to therapy may be due to differences in the levels of cytokines. Thus, targeting the appropriate cytokines has the potential to provide effective treatment. Treatments currently available or under development for atopic dermatitis include anti-IL-5, anti-IL-23, anti-IL-22, anti-OX40, anti-IL-4Rα, anti-IL-13, anti-TSLP, and anti-IL-33. The present invention provides for the measurement of biomarker polypeptides, such as BDNF and amphiregulin, that can act as proxies for cytokines, such as TSLP that are much more difficult to measure.

### Biomarkers

In particular embodiments, a biomarker is an organic biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease.

In one aspect, the invention provides a panel of biomarkers that are differentially present in tissues (e.g., blood, plasma, sera, skin samples) of atopic dermatitis subjects responsive to anti-TSLP therapy. Accordingly, a panel of biomarkers includes two or more of the following: Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 3 (NTRK3), Brain Derived Neurotrophic Factor (BDNF), Neurotrophic Tyrosine Kinase Receptor Type 2 (NTRK2); and amphiregulin (AREG). In a particular embodiment, the panel includes CNTF and BDNF. In another embodiment, a panel includes BDNF and Amphiregulin. In another embodiment, a panel includes BDNF, NTRK3, Amphiregulin, TSLPR/CRLF2, CNTF, NTF3, NTF4 or combinations thereof. In another aspect, the invention provides a panel of capture reagents that specifically bind the biomarkers that are differentially present in atopic dermatitis subjects responsive to anti-TSLP therapy.

The invention provides panels comprising isolated biomarkers. The biomarkers can be isolated from biological fluids, such as blood or blood serum or other biological sample, such as skin biopsy. They can be isolated by any method known in the art, including using a capture reagent or probe that specifically binds the biomarker. In certain embodiments, this isolation is accomplished using the mass and/or binding characteristics of the markers. For example, a sample comprising the biomolecules can be subject to chromatographic fractionation and subject to further separation by, e.g., acrylamide gel electrophoresis. Knowledge of the identity of the biomarker also allows their isolation by immunoaffinity chromatography. By “isolated biomarker” is meant at least 60%, by weight, free from proteins and naturally-occurring organic molecules with which the marker is naturally associated. Preferably, the preparation is at least 75%, more preferably 80, 85, 90 or 95% pure or at least 99%, by weight, a purified marker.

The biomarkers of the invention can be detected by any suitable method. The methods described herein can be used individually or in combination for a more accurate detection of the biomarkers (e.g., biochip in combination with mass spectrometry, immunoassay in combination with mass spectrometry, and the like). A biomarker of the invention may be detected in a biological sample of the subject (e.g., tissue, fluid), including, but not limited to, blood, blood serum or tissue sample (e.g., a skin biopsy), a cell isolated from a patient sample, and the like.

Detection paradigms that can be employed in the invention include, but are not limited to, optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described infra.

### Detection by Immunoassay

In particular embodiments, the biomarkers of the invention are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In aspects of the invention, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In aspects of the invention, a sample is analyzed by means of a protein biochip (also known as a protein microarray). Such biochips are useful in high-throughput low-cost screens to identify alterations in the expression or post-translation modification of a polypeptide of the invention, or a fragment thereof. In embodiments, a protein biochip of the invention binds a biomarker present in a subject sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont. Calif.), Zyomyx (Hayward. Calif.), Packard BioScience Company (Meriden, Conn.), Phylos (Lexington, Mass.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In other embodiments, wash steps will occur at 68 C in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Diagnostic Methods

The present invention provides methods of stratifying atopic dermatitis patients for treatment with an anti-TLSP therapy (e.g., Tezepelumab), anti-IL-33 therapy, anti-ST2 therapy (receptor for IL-33), and/or predicting and/or determining response to anti-TSLP therapy in patients having atopic dermatitis (AD). As described herein, it has been discovered that altered levels, expression, or activity in one or more of the biomarkers Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), Neurotrophic Tyrosine Kinase Receptor Type 3 (NTRK3), Amphiregulin, and/or Brain Derived Neurotrophic Factor (BDNF) is indicative of TSLP-mediated atopic dermatitis (AD) in subjects having AD. Such diagnostic methods are useful for determining responsiveness to anti-TSLP therapy and informing subject treatment.

### Therapeutic Methods

The present invention provides methods of treating atopic dermatitis, or symptoms thereof, by administering an agent that decreases TSLP levels, expression, or biological activity. An agent that inhibits TSLP biological activity or expression is provided to a subject having atopic dermatitis in a pharmaceutical composition, where the pharmaceutical composition comprises an effective amount of the agent and a suitable excipient. In one embodiment, the agent is an anti-TSLP antibody that decreases the level, expression, or activity of TSLP polypeptide in a subject. Anti-TSLP antibodies are known in the art and include Tezepelumab. While methods of atopic dermatitis treatment vary depending on the characterization of AD, anti-TSLP therapy will be used in patients identified as responsive to such treatment. As used herein the disclosure relating to “therapeutic methods” equally applies to the use of a compound for the manufacture of a medicament for the treatment of a disease, as well as to a compound for use in the treatment of a disease.

### Anti-TSLP Antibodies

Atopic dermatitis subjects responsive to treatment with an anti-TSLP antibody are identified by characterizing the level, expression, or activity of one or more biomarkers of the invention in the subject. Once selected for treatment, such subjects may be administered virtually any anti-TSLP antibody known in the art. Suitable anti-TSLP antibodies include, for example, known anti-TSLP antibodies, commercially available anti-TSLP antibodies, anti-TSLPR antibodies, or anti-TSLP antibodies developed using methods well known in the art. An exemplary anti-TSLP antibody is Tezepelumab (see U.S. Pat. Nos. 7,982,016; 8,163,284; 9,284,372).

Antibodies useful in the invention include immunoglobulins, monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies disclosed herein), intrabodies, and epitope-binding fragments of any of the above. In particular, antibodies include immunoglobulin molecules and immunologically active fragments of immunoglobulin molecules, e.g., molecules that contain at least one antigen-binding site.

Anti-TSLP antibodies encompass monoclonal human, humanized or chimeric anti-TSLP antibodies. Anti-TSLP antibodies used in compositions and methods of the invention can be naked antibodies, immunoconjugates or fusion proteins. In certain embodiments, an anti-TSLP antibody is a human, humanized or chimeric antibody having an IgG isotype, particularly an IgG1, IgG2, IgG3, or IgG4 human isotype or any IgG1, IgG2, IgG3, or IgG4 allele found in the human population. Antibodies of the human IgG class have advantageous functional characteristics, such as a long half-life in serum and the ability to mediate various effector functions (Monoclonal Antibodies: Principles and Applications, Wiley-Liss, Inc., Chapter 1 (1995)). The human IgG class antibody is further classified into the following 4 subclasses: IgG1, IgG2, IgG3 and IgG4. The IgG1 subclass has the high ADCC activity and CDC activity in humans (Chemical Immunology, 65, 88 (1997)). In other embodiments, an anti-TSLP antibody is an isotype switched variant of a known anti-TSLP antibody.

### Kits

The invention provides kits for the treatment of atopic dermatitis (AD). In one embodiment, the invention provides kits for characterizing the responsiveness of a subject having atopic dermatitis to anti-TSLP treatment. A diagnostic kit of the invention provides a reagent (e.g., primers/probes for and housekeeping reference genes) for measuring the expression, level, or activity of a polypeptide or nucleic acid molecule biomarker of the invention. If desired, the kit further comprises instructions for measuring the level, expression, or activity of a biomarker of the invention and/or instructions for administering an anti-TSLP therapy to a subject having AD.

In a further embodiment, the kit may also include an agent that reduces the level, expression, or activity of a TSLP polynucleotide or polypeptide, such as an anti-TSLP antibody (e.g., Tezepelumab). In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments. If desired, the agent is provided together with instructions for administering the agent to a subject having atopic dermatitis.

In particular embodiments, the instructions include at least one of the following: description of the therapeutic agent; dosage schedule and administration for treatment of atopic dermatitis or symptoms thereof; precautions; warnings indications; counter-indications over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Identification of Novel Biomarkers of Response to Tezepelumab in Atopic Dermatitis Patients

In a small trial, skin disease severity was assessed on enrolled subjects using the eczema area severity index (EASI). Improvement was observed in patients treated with Tezepelumab. Following treatment with either placebo or Tezepelumab, subjects were classified as responders if there was a 50% reduction in EASI scores (compared to baseline) at 2 or more time points in the study. Serum samples were collected from 12 patients with moderate to severe atopic dermatitis on Day 1, Day 29 and Day 85. Subjects were treated i.v. with either Tezepelumab (700 mg; n=9) or placebo (n=3) at baseline. Peripheral blood was collected for proteomic analysis. RNA was obtained from lesional and non-lesional skin biopsies before and after treatment with either placebo or Tezepelumab (700 mg).

Four patients receiving Tezepelumab achieved an EASI50 (50% improvement in skin disease) at 2 or more time points during the trial. The serological samples from patients treated with AMG 157 were analyzed to determine if there was a difference in the level of neurotrophic factors in responders and non-responders, which could serve as biomarkers to indicate responsiveness to therapy with Tezepelumab.

Sera from all subjects were evaluated using the previously described SOMAscan proteomic assay (Gold et al., 2010, PLOS One 5 (12):e15004. Rohloff et al., 2014, Molecular Therapy-Nucleic Acids 3: e201). Briefly, the version of the SOMAscan proteomic assay utilized in these studies measured 1,129 proteins using modified aptamers that target each protein. Protein concentrations in sera were transformed into a corresponding signature of DNA aptamer concentrations and then quantified on a DNA microarray. SOMAscan data are reported in relative fluorescence unites (RFU). To reduce heteroscedasticity, RFU data were log 2 transformed prior to statistical analysis.

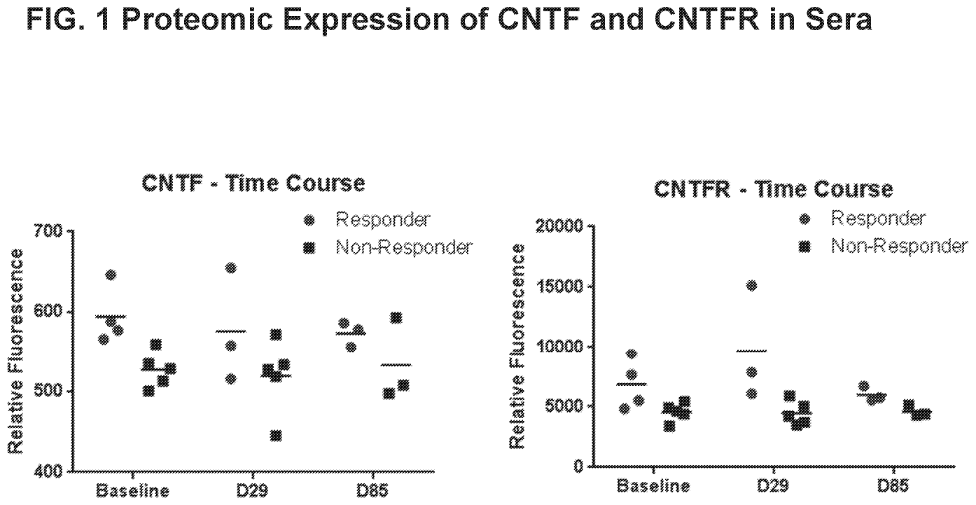

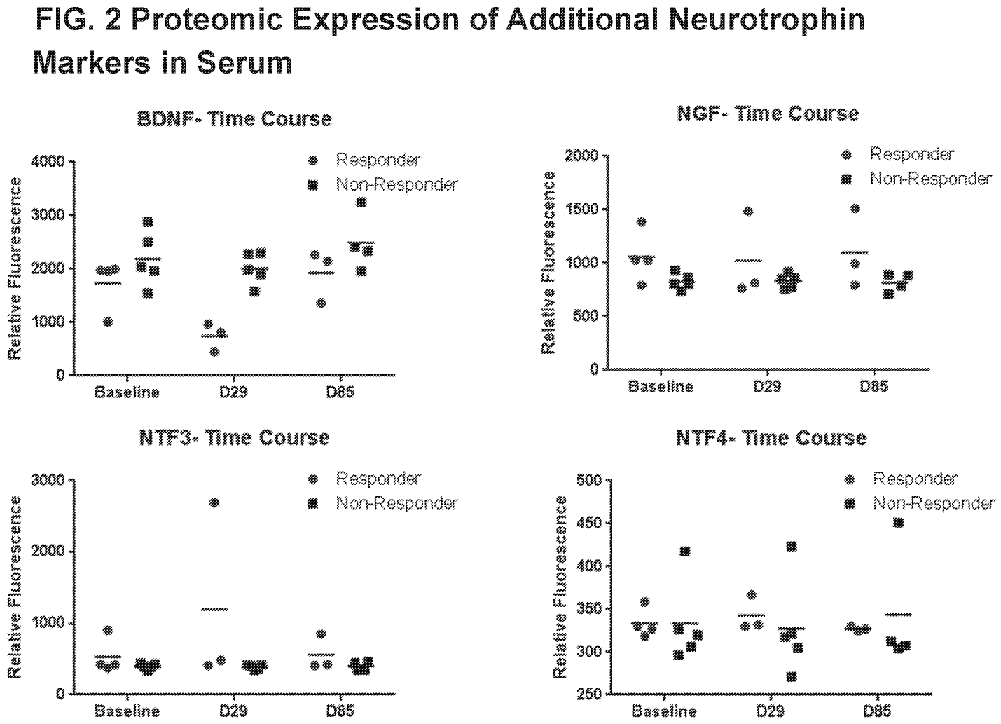

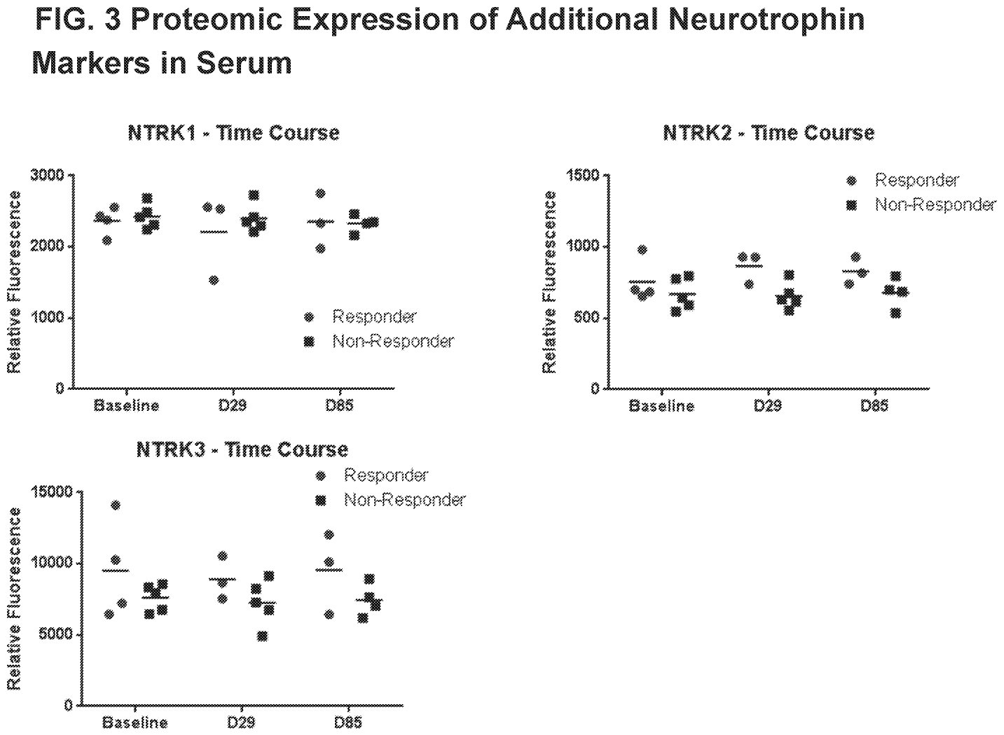

Treatment with TEZEPELUMAB was associated with elevated serum CNTF and CNTFR at baseline in “responders.” (FIG. 1). Thus, CNTF and CNTFR were identified as differentially expressed in responders and non-responders. Proteomic expression L levels of Brain Derived Neurotrophin (BDNF), Nerve Growth Factor (NGF), NTF3, and NTF4 were also characterized (FIG. 2). Subjects that responded to TEZEPELUMAB showed a reduction in levels of BDNF in serum relative to non-responders. Levels of BDNF in sera were dramatically reduced by day 29 in responding patients. This is of particular interest given that BDNF and TSLP levels correlate with levels of eosinophil survival. Thus, as BDNF levels are reduced, eosinophil survival is expected to decrease. Neurotrophic Tyrosine Kinase Receptor Type 1 (NTRK1), Neurotrophic Tyrosine Kinase Receptor Type 2 (NTRK2), Neurotrophic Tyrosine Kinase Receptor Type 3 (NTRK3) proteomic expression in sera was also characterized (FIG. 3. No significant changes were observed.

### Example 2: Identification of Nucleic Acid Biomarkers of Response to Tezepelumab in Atopic Dermatitis Patients

Genomic expression of additional neutrophin markers in lesional and non-lesional skin biopsies was also examined. Lesional and non-lesional skin samples from the atopic dermatitis cohort were analyzed for levels of neurotrophic factors. Skin biopsies were collected from lesional and non-lesional skin of atopic dermatitis patients on Day 1 and Day 29. Skin biopsies (6 mm) were cut longitudinally and one half was placed into liquid nitrogen. The frozen biopsies were then maintained at −70° C. or in dry ice. RNA was isolated from the samples frozen in liquid nitrogen. Messenger RNA was analyzed by microarray using Nugen Ovation cDNA labeling kits and Affymetrix HT_HG-U133_Plus_PM microarrays.

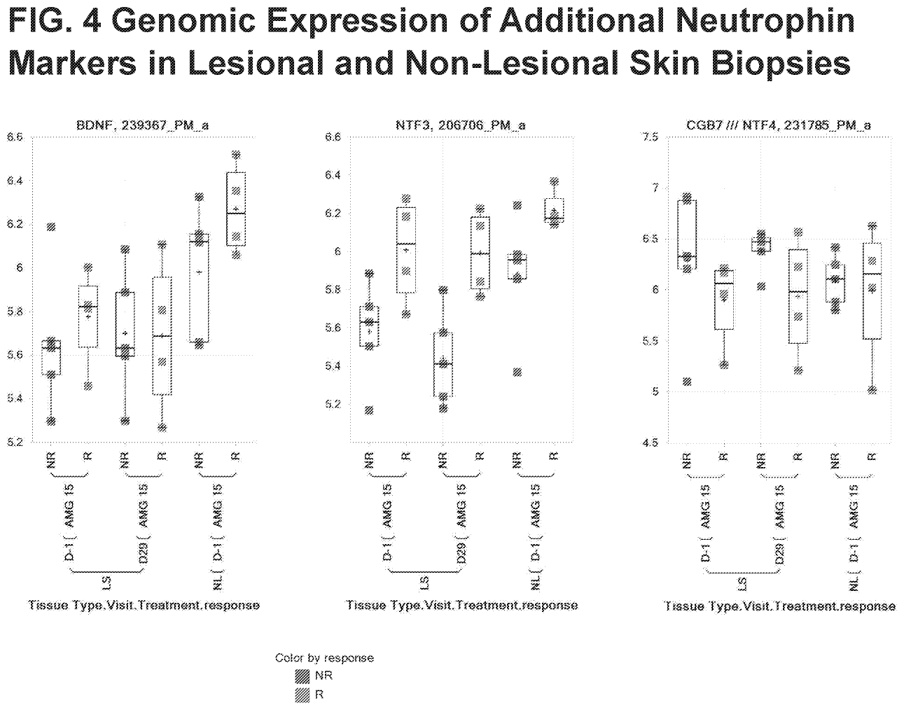

Additional neutrophin markers are analyzed for gene expression in lesional and non-lesional skin biopsies included Amphiregulin, CNTF, CNTFR, BDNF, NTF3, NTF4, NGF, NTRK1, NTRK2, and NTRK3. BDNF genomic expression levels were elevated at baseline in both lesional skin and non-lesional skin samples of patients characterized as responsive to anti-TLSP therapy relative to levels present in non-responders (FIG. 4). NTF3 genomic expression level in skin is also increased at baseline in anti-TLSP therapy responders vs. non-responders (FIG. 4).

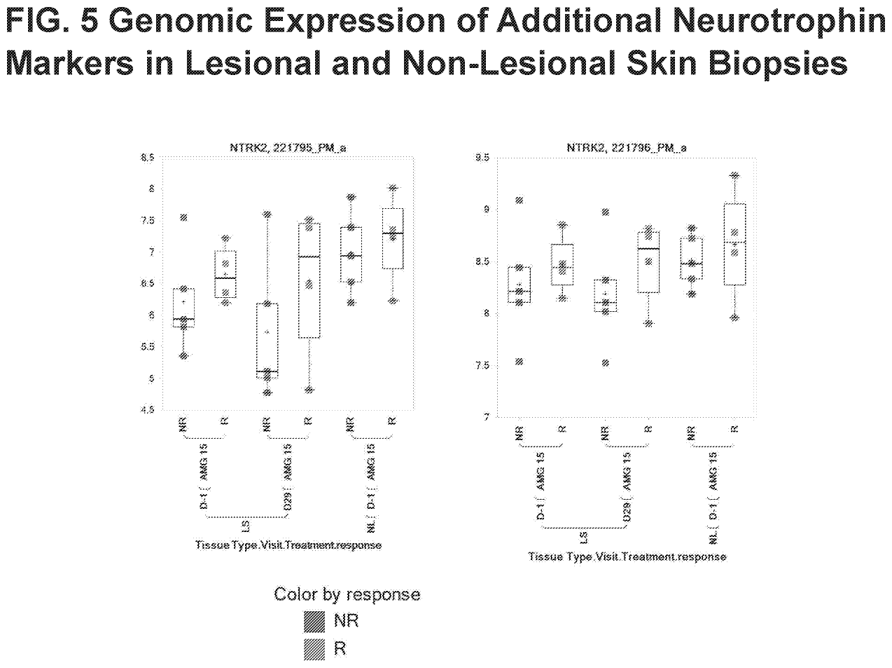

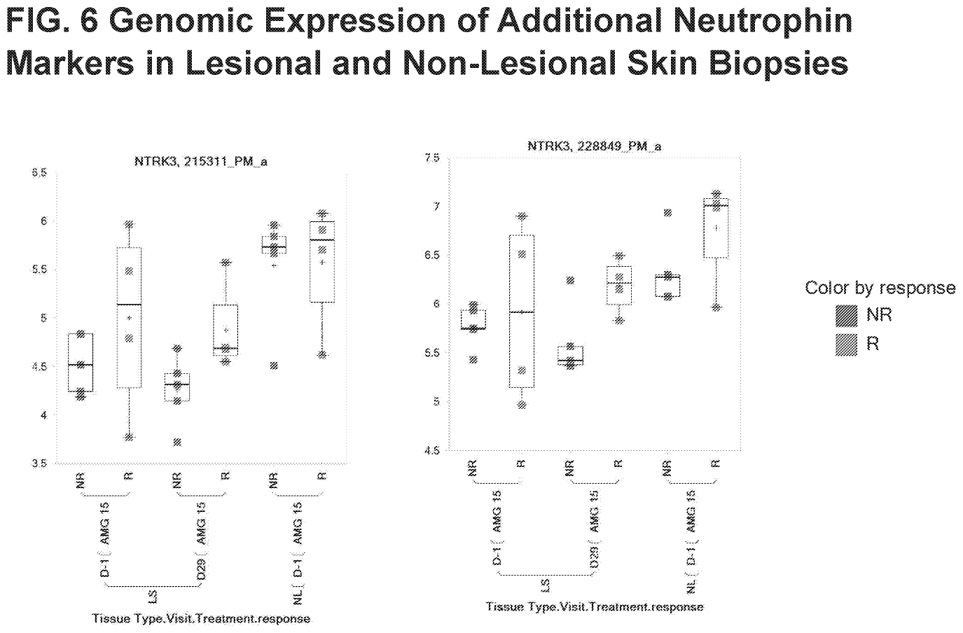

NTRK2 genomic expression levels were elevated at baseline in lesional skin of subjects subsequently found to respond to anti-TLSP therapy relative to levels of genomic expression present in non-responders (FIG. 5). NTRK3 genomic expression was also elevated in lesional and non-lesional skin at baseline of subjects subsequently found to respond to anti-TLSP therapy relative to levels present in corresponding sample obtained from non-responders (FIG. 6).

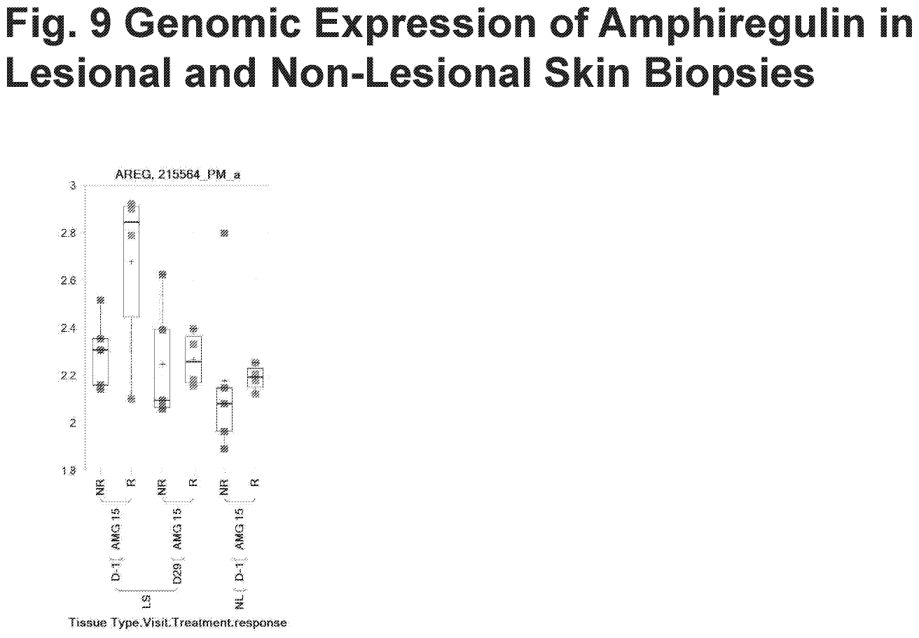

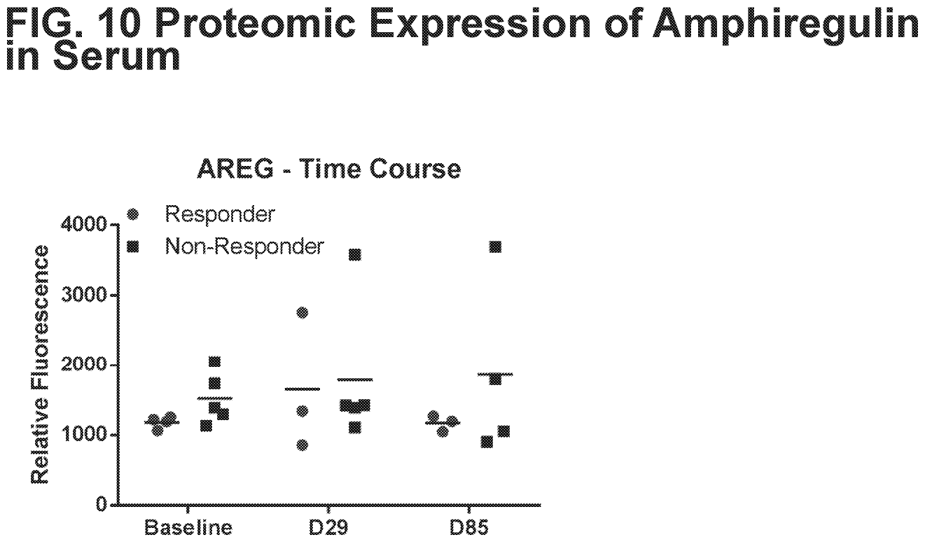

Amphiregulin genomic expression levels were elevated at baseline in lesional and non-lesional skin samples of subjects subsequently found to respond to anti-TLSP therapy relative to levels of genomic expression present in corresponding samples obtained from non-responders (FIG. 9). Interestingly, proteomic expression of amphiregulin was reduced at baseline in serum samples obtained from subjects subsequently found to respond to anti-TLSP therapy relative to levels present in corresponding samples obtained from non-responders (FIG. 10).

### Example 3: Correlation of Selected Biomarker Expression in Moderate to Severe Atopic Dermatitis

Serological samples were collected from healthy controls with no history of skin disease and moderate to severe atopic dermatitis subjects. Subjects were recruited through protocols with TR Bio (20 healthy controls; 41 atopic dermatitis) and a collaboration with Dr. Emma Guttman-Yassky at Mount Sinai School of Medicine (20 healthy controls; 35 atopic dermatitis).

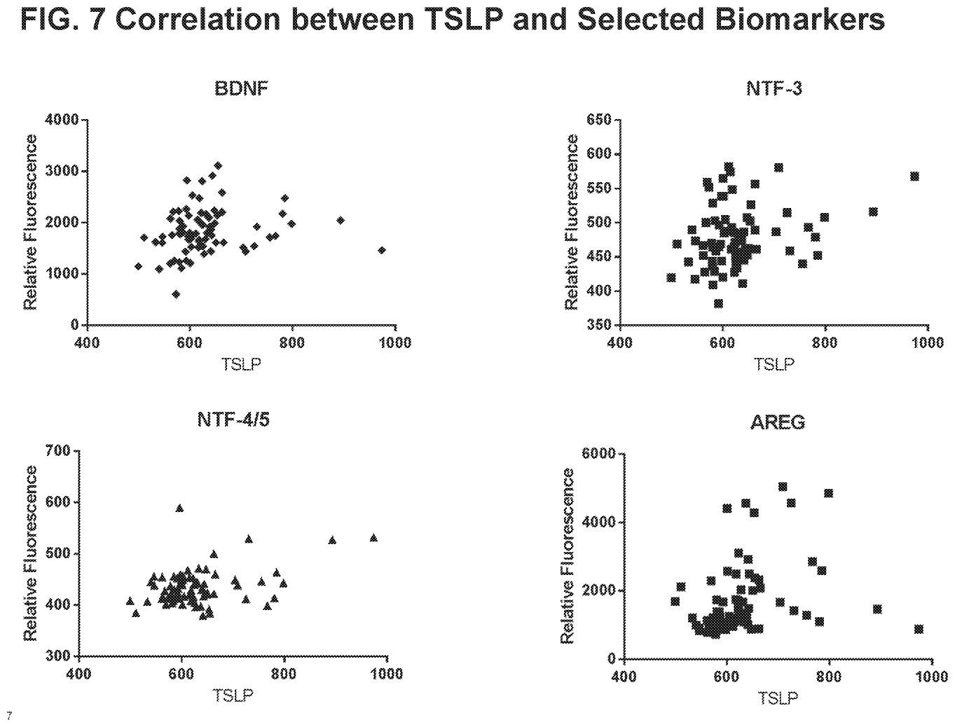

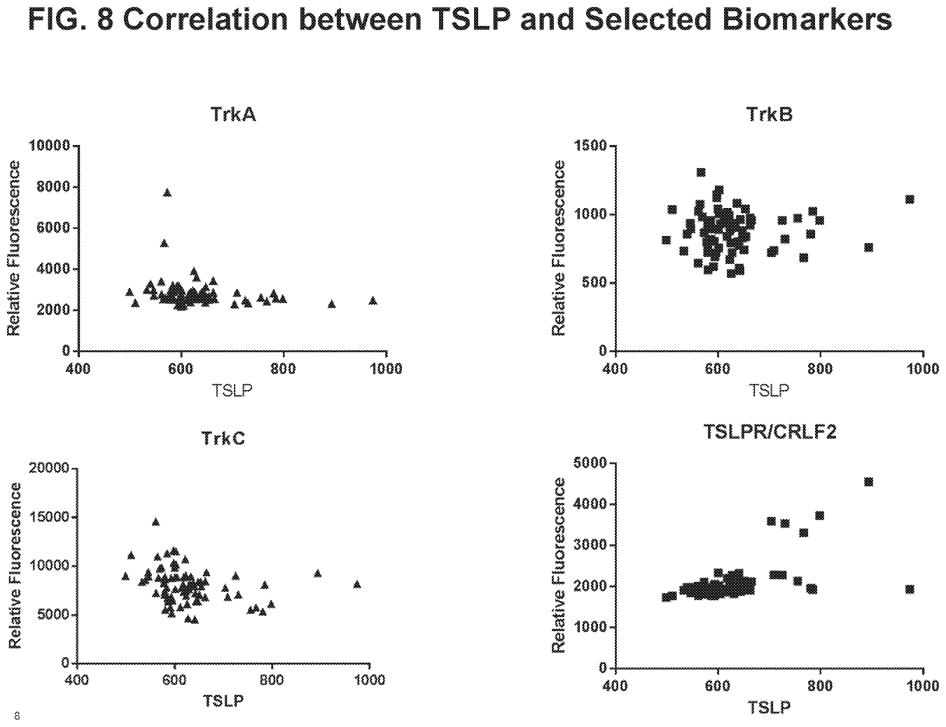

Sera from all subjects were evaluated using the previously described SOMAscan proteomic assay described above in Example 2. Selected biomarkers were evaluated for correlations with TSLP measurements from the same subjects (Table 1: FIGS. 7 and 8). A statistically significant correlation was observed between protein levels of TSLP and BDNF (FIG. 7), as well as between protein levels of TSLP and Amphiregulin. A correlation was also observed between protein levels of TSLP and the TSLP receptor, CRLF2 (FIG. 8).

TABLE 1
Correlation between TSLP and selected biomarkers

| Protein | R value | P value |
|---|---|---|
| BDNF | 0.2651 | 0.0207 |
| NTF3 | 0.2056 | 0.0747 |
| NTF4 | 0.1988 | 0.0852 |
| CNTF | n/a | n/a |
| CNTFRa | −0.4317 | <0.0001 |
| AREG | 0.4252 | 0.0001 |
| TrkA | −0.2800 | 0.0143 |
| TrkB | −0.0168 | 0.8855 |
| TrkC | −0.3490 | 0.0020 |
| TSLPR | 0.5130 | <0.0001 |

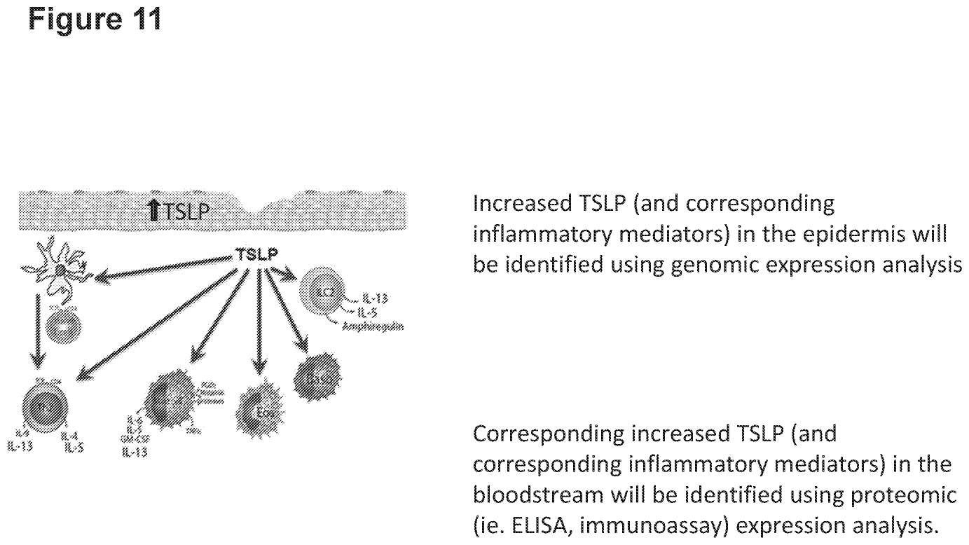

A model for how increased levels of TLSP in skin affects marker levels in skin and circulation is provided at FIG. 11.

### Example 4: Induction of Selected Biomarker Expression in Eosinophils and Basophils

Purified eosinophil (Eol-1) and basophil (KU812) cell lines were purchased and cultured in RPMI media supplemented with 10% fetal bovine serum. Cells were seeded at 2.5×10⁵/well in flat-bottomed 96 well microculture plates and stimulated with 50 ng/ml of rhTSLP (Peprotech) for 24 hours. Post-stimulation, cells were collected and suspended in miRVana Lysis/Binding buffer and total RNA extracted using the mirVana miRNA Isolation Kit (Life Technologies). RNA purity and concentration were determined spectrophotometrically. 100 ng of total RNA was reverse transcribed to cDNA using SuperScript III reverse transcriptase and random hexamers (Invitrogen). The resulting cDNA was pre-amplified using TaqMan PreAmp Master Mix and a primer pool of TaqMan assays for genes of interest (Life Technologies). After pre-amplification, amplified samples were diluted 1:4 in DNA Suspension Buffer (TEKnova, Hollister, Calif.) and held at −20° C. or used immediately for PCR. Real-time was performed with the Biomark HD system and 48.48 dynamic arrays (Fluidigm). Delta Ct values (ΔCt) were calculated using the mean of two reference genes (GAPDH, ACTB). Fold change values were determined by calculating 2^{-ΔΔCt} using expression of genes of interest in unstimulated cells as the control.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of treating a subject having atopic dermatitis, the method comprising administering to the subject an agent that reduces the expression or activity of a Thymic Stromal Lymphopoietin (TSLP) polypeptide, wherein the subject is identified as having an increase in the level of Brain Derived Neurotrophin (BDNF) polypeptide in circulation or an increase in the level of Brain Derived Neurotrophin (BDNF) polynucleotide in a skin sample derived from the subject relative to a reference.

2. The method of claim 1, wherein BDNF polypeptide in circulation is measured in blood, plasma, or serum sample derived from the subject.

3. The method of claim 1, wherein the method further comprises detecting an increase in Ciliary Neurotrophic Factor (CNTF) polynucleotide or Ciliary Neurotrophic Factor Receptor (CNTFR) polynucleotide in circulation.

4. The method of claim 1, wherein BDNF polynucleotide in skin is increased in a skin biopsy of lesional or non-lesional skin.

5. The method of any one of claims 1-4, wherein the method further comprises detecting an increase in amphiregulin polynucleotide in lesional and non-lesional skin biopsies.

6. The method of any one of claims 1-5, wherein the method further comprises detecting an increase in a polynucleotide biomarker selected from the group consisting of Neurotrophic Tyrosine Kinase Receptor Type 2 (NTRK2), Neurotrophic Tyrosine Kinase Receptor Type 3 (NTRK3), and Neurotrophin Factor 3 (NTF3).

7. The method of any one of claims 1-5, wherein the polypeptide is detected in an immunological assay.

8. The method of any one of claims 1-5, wherein the polynucleotide is detected by hybridization to a microarray or by gene expression analysis.

9. A method of treating a subject having atopic dermatitis, the method comprising administering to the subject an agent that reduces the expression or activity of a Thymic Stromal Lymphopoietin (TSLP) polypeptide, wherein the subject is identified as having an increase in the level of Brain Derived Neurotrophin (BDNF) polynucleotide and Amphiregulin polynucleotide in a skin sample derived from the subject relative to a reference.

10. A method of treating a subject having atopic dermatitis, the method comprising administering to the subject an agent that reduces the expression or activity of a Thymic stromal lymphopoietin (TSLP) polypeptide, wherein the subject is identified as having an increase in the level of Brain Derived Neurotrophin (BDNF) polynucleotide, Amphiregulin polynucleotide, and one or more of NTRK2, NTRK3, or NTF3 polynucleotides in a skin sample derived from the subject relative to a reference.

11. The method of claim 9 or 10, wherein the polynucleotide is detected by hybridization to a microarray.

12. A method of treating a subject having atopic dermatitis, the method comprising administering to the subject an agent that reduces the expression or activity of a Thymic stromal lymphopoietin (TSLP) polypeptide, wherein the subject is identified as having an increase in the level of Brain Derived Neurotrophin (BDNF) polypeptide and an increase in Ciliary Neurotrophic Factor (CNTF) and/or Ciliary Neurotrophic Factor Receptor (CNTFR) in blood, plasma, or sera derived from the subject relative to a reference.

13. The method of claim 12, wherein the polypeptide is detected in an immunological assay.

14. A method of treating a subject having atopic dermatitis, the method comprising administering to the subject an agent that reduces the expression or activity of a Thymic stromal lymphopoietin (TSLP) polypeptide, wherein the subject is identified as having an alteration in a biomarker polypeptide selected from the group consisting of Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3) in a blood, plasma, or sera sample of the subject relative to a reference, thereby treating the atopic dermatitis.

15. A method of treating a subject having atopic dermatitis, the method comprising administering to the subject an agent that reduces the expression or activity of a Thymic stromal lymphopoietin (TSLP) polypeptide, wherein the subject is identified as having an alteration in a biomarker polynucleotide selected from the group consisting of Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3) in a skin sample of the subject relative to a reference, thereby treating the atopic dermatitis.

16. The method of any one of claims 1-15, wherein the atopic dermatitis is responsive to treatment with the agent that reduces the expression or activity of a Thymic stromal lymphopoietin (TSLP) polypeptide.

17. The method of any one of claims 1-15, wherein the agent that reduces the expression or activity of the TSLP polypeptide is an anti-TSLP antibody, or antigen binding portion thereof.

18. The method of any one of claims 1-15, wherein the antibody, or antigen binding portion thereof, comprises:
(a) a heavy chain variable region CDR1 comprising the amino acid sequence of SEQ ID NO: 6;
(b) a heavy chain variable region CDR2 comprising the amino acid sequence of SEQ ID NO: 7;
(c) a heavy chain variable region CDR3 comprising the amino acid sequence of SEQ ID NO: 8;
(d) a light chain variable region CDR1 comprising the amino acid sequence of SEQ ID NO: 3;
(e) a light chain variable region CDR2 comprising the amino acid sequence of SEQ ID NO: 4; and
(f) a light chain variable region CDR3 comprising the amino acid sequence of SEQ ID NO: 5

19. The method of any one of claims 1-15, wherein the antibody, or antigen binding portion thereof, comprises the heavy chain sequence of SEQ ID NO: 10 and the light chain sequence of SEQ ID NO 12.

20. The method of any one of claims 1-15, wherein the antibody is Tezepelumab.

21. The method of any one of claims 1-20, wherein the subject is human.

22. The method of any one of claims 1-21, wherein the reference is the level, expression, or activity of the corresponding polypeptide or nucleic acid molecule biomarker present in a control sample.

23. The method of claim 22 wherein the control sample is derived from a subject having atopic dermatitis that is not responsive to anti-TSLP therapy.

24. The method of claim 22 or 23 wherein the control sample is derived from a healthy subject.

25. A method of identifying a subject as having atopic dermatitis (AD) responsive to an anti-TSLP therapy, the method comprising detecting an increase in the level of Brain Derived Neurotrophic Factor (BDNF) polypeptide in circulation or an increase in the level of Brain Derived Neurotrophic Factor (BDNF) polynucleotide in a skin sample derived from the subject relative to a reference, thereby identifying the subject as having atopic dermatitis that is responsive to anti-TSLP therapy.

26. A method of identifying a subject as having atopic dermatitis (AD) responsive to an anti-TSLP therapy, the method comprising detecting an increase in the level of Brain Derived Neurotrophic Factor (BDNF) polynucleotide and Amphiregulin polynucleotide in a skin sample derived from the subject relative to a reference, thereby identifying the subject as having atopic dermatitis that is responsive to anti-TSLP therapy.

27. A method of identifying a subject as having atopic dermatitis (AD) responsive to an anti-TSLP therapy, the method comprising detecting an increase in the level of Brain Derived Neurotrophic Factor (BDNF) polynucleotide, Amphiregulin polynucleotide, and one or more of NTRK2, NTRK3, or NTF3 polynucleotides in a skin sample derived from the subject, thereby identifying the subject as having atopic dermatitis that is responsive to anti-TSLP therapy.

28. A method of identifying a subject as having atopic dermatitis (AD) responsive to an anti-TSLP therapy, the method comprising detecting an increase in the level of Brain Derived Neurotrophic Factor (BDNF) polypeptide and an increase in CNTF and/or CNTFR in blood, plasma, or sera derived from the subject, thereby identifying the subject as having atopic dermatitis that is responsive to anti-TSLP therapy.

29. A method of identifying a subject as having atopic dermatitis (AD) responsive to an anti-TSLP therapy, the method comprising
(a) detecting an antibody binding to a circulating polypeptide marker selected from the group consisting of Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3) in a blood, plasma, or sera sample of the subject; and
(b) detecting an alteration in the level of said marker in the sample relative to a reference, thereby identifying the subject as having atopic dermatitis (AD) responsive to an anti-TSLP therapy.

30. A method of identifying a subject as having atopic dermatitis (AD) responsive to an anti-TSLP therapy, the method comprising
(a) detecting a probe binding to a polynucleotide marker selected from the group consisting of Amphiregulin (AREG), Brain Derived Neurotrophin (BDNF), Ciliary Neurotrophic Factor (CNTF), Ciliary Neurotrophic Factor Receptor (CNTFR), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), and Neurotrophic Tyrosine Kinase Receptor Type 3 (NTRK3) in a skin sample of the subject; and
(b) detecting an alteration in the level of said marker in the sample relative to a reference, thereby identifying the subject as having atopic dermatitis (AD) responsive to an anti-TSLP therapy.

31. A method of monitoring the efficacy of therapy in a subject, the method comprising
(a) administering an anti-TSLP therapy to the subject; and
(b) detecting the level of Brain Derived Neurotrophic Factor polynucleotide in a skin sample derived from the subject relative to the level of Brain Derived Neurotrophic Factor polynucleotide in a skin sample obtained from the subject at an earlier point in time, wherein a decrease in the level of BDNF over time indicates that the anti-TSLP therapy is effective.

32. The method of claim 31, wherein the method further comprises detecting the level of amphiregulin polypeptide in the sera of the subject relative to the level present in the sera of the subject at an earlier point in time, wherein an increase in said level over time indicates that the anti-TSLP therapy is effective.

33. A kit for the treatment of atopic dermatitis (AD), the kit comprising an agent that reduces the expression or activity of a Thymic stromal lymphopoietin (TSLP) polypeptide, and one or more of a capture molecule or probe that specifically binds a polypeptide or polynucleotide biomarker selected from the group consisting of Amphiregulin (AREG), Ciliary Neurotrophic Factor (CNTF), Ciliary Neurotrophic Factor Receptor (CNTFR), Brain Derived Neurotrophin (BDNF), Neurotrophin 3 (NTF3), Neurotrophin 4 (NTF4), Nerve Growth Factor (NGF), Neurotrophic Tyrosine Kinase Receptor Type 1 (NTRK1), Neurotrophic Tyrosine Kinase Receptor Type 2 (NTRK2), Neurotrophic Tyrosine Kinase Receptor Type 3 (NTRK3).
